(19) United States
(12) Plant Patent Application Publication
(10) Pub. No.: US 2014/0150143 P1
(43) Pub. Date: May 29, 2014

(54) Blueberry plant, EB 8-38

(50) ;

(71) Applicant: Vincent Mazzardis, Joondalup (AU)

(72) Inventor: Vincent Mazzardis, Joondalup (AU)

(73) Assignees: Rolfe Nominees Pty Ltd, Crows Nest (AU); Prunus Persica Pty Ltd, Joondalup (AU)

(21) Appl. No.: 13/998,669

(22) Filed: Nov. 21, 2013

(30) Foreign Application Priority Data
Nov. 28, 2012 (AU) 2012/258

(51) Int. Cl. A01H 5/00

(52) U.S. Cl. PLT/157

(57) ABSTRACT

A new and distinct variety of blueberry plant, which is denominated varietally as ‘EB 8-38’ and which produces an attractively colored, extra large fruit, having a very good fruit flavor and earlier flowering and fruit production dates when grown under the ecological conditions prevailing in Yanchep Springs, Western Australia.

### LATIN NAME

‘Vaccinium Hybrid’

### VARIETAL DENOMINATION

‘EB 8-38’

### RELATED APPLICATION DATA

The presented patent application claims priority Australian Plant Breeder Rights Application Serial No. 2012/258 and which was filed on Nov. 28, 2012.

### BACKGROUND OF THE NEW VARIETY

The present invention relates to a new, novel, and distinct variety of blueberry plant ‘Vaccinium Hybrid,’ and which has been denominated varietally, as ‘EB 8-38’, hereinafter.

### ORIGIN AND ASEXUAL REPRODUCTION OF THE NEW VARIETY

The present variety of blueberry plant resulted from an ongoing program of plant breeding. The purpose of this program is to improve the commercial quality of various plant varieties by creating and releasing promising selections of plants including blueberries. To this end, I have made both controlled and hybrid cross-pollinations each year in order to produce resulting plant populations from which improved progenies are evaluated and selected.

The blueberry plant ‘EB 8-38’ was originated by me and selected from a population of new plants growing on my farm, and which is located near Yanchep Springs, Western Australia. The new plant was derived from a controlled pollination, which was conducted by me, using the seed parent ‘BB-1’ [unpatented], and the pollen parent ‘99-1’ [also unpatented] during the 2005 growing season on my farm which is located in Yanchep Springs, Western Australia. The seed parent ‘BB-1’ is characterized by a semi-spreading growth habit, and further has an early season flowering date. The seed parent also produces medium sized fruit. In contrast, the pollen parent has a spreading growth habit, which produces early season flowers, and which further produces fruit which are relatively large in size for the species. Seed derived from this aforementioned cross-pollination was collected from the seed parent (‘BB-1’) and produced approximately 500 plants. These plants were subsequently grown and the first fruit produced from these plants were seen and studied in 2007.

Further, the same plants derived from this original cross-pollination were again observed and considered during the 2008 growing season. During this 2008 assessment, the new variety ‘EB 8-38’ was selected for further asexual reproduction. The new variety ‘EB 8-38’ was subsequently asexually reproduced by cuttings and those progeny were later evaluated during the 2009 through 2012 growing seasons. The subsequent observations of these asexually reproduced plants led to the conclusion that the new variety ‘EB 8-38’ was a distinct and novel variety of blueberry plant and had characteristics true to the original plant. The present variety is particularly novel and characterized by producing extra large sized fruit, and further has a dry picking scar, a very good fruit flavor, and earlier flowering and fruit production times in relative comparison to the closest known varieties.

In this regard, and when compared against the closest known variety, that being the ‘Sharpeblue’ blueberry plant [unpatented], the present variety has a date of fruit maturity which is very early in the growing season, and further produces very large fruit. Additionally, the new variety has an intermediate to spreading growth pattern, and further produces oblate shaped fruit. In contrast, the ‘Sharpeblue’ blueberry plant has fruit which are mature for harvesting and shipment in the early to mid season, Further the fruit size of the variety “Sharpeblue' is medium in size as compared to other common varieties. Again, the ‘Sharpeblue’ blueberry plant has an intermediate growth pattern, which is distinguishable from the new variety.

### BRIEF DESCRIPTION OF THE DRAWING

The accompanying drawings, which are provided, are color photographs of the new blueberry plant.

FIG. 1 depicts several fruit of the new variety, which are sufficiently matured for harvesting and shipment; and a vegetative shoot bearing typical leaves.

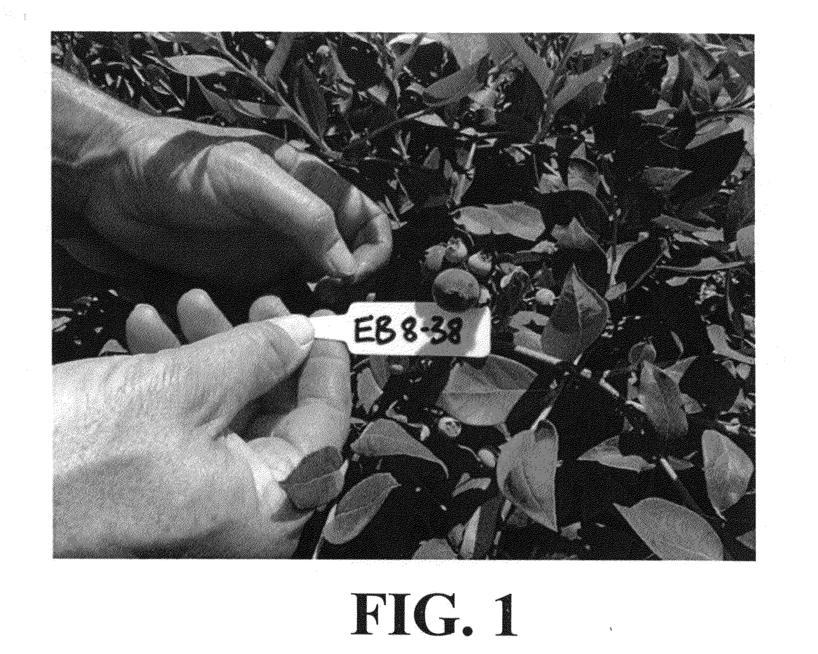

FIG. 2 shows several fruit which are sufficiently matured for harvesting at shipment and a twig bearing typical leaves.

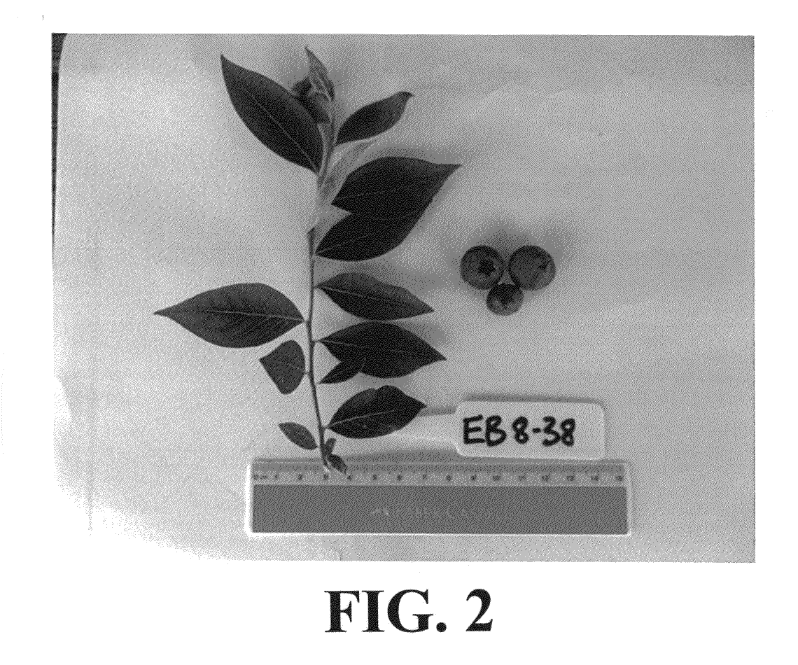

FIG. 3 shows the typical growth habit of the new variety of blueberry plant.

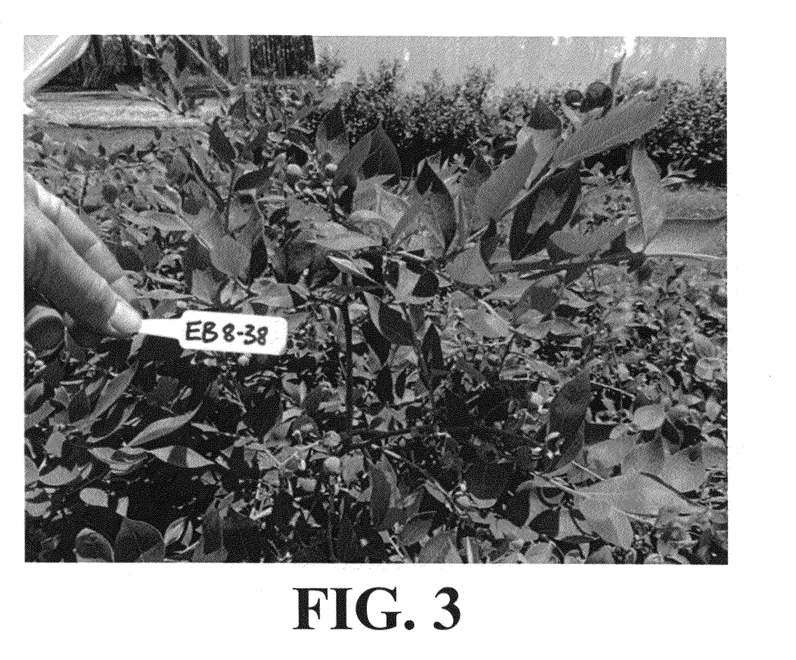

The colors in these photographs are as nearly true as is reasonably possible in a color representation of this type. Due to chemical development, processing and printing, the leaves and fruit depicted in these photographs may or may not be accurate when compared to the actual specimen. For this reason, future color references should be made to the common color names as provided, hereinafter.

### NOT A COMMERCIAL WARRANTY

The following detailed description has been prepared to solely comply with the provisions of 35 U.S.C. §112, and does not constitute a commercial warranty (either express or implied) that the present variety will, in the future, display the botanical, horticultural, or other characteristics as set forth, hereinafter. Therefore, this disclosure may not be relied upon to support any future legal claims, including, but not limited to, breach of warranty of merchantability, or fitness for any particular purpose, or non-infringement, which is directed, in whole, or in part, to the present new variety.

### DETAILED DESCRIPTION

Referring more specifically to the botanical and horticultural details of this new and distinct variety of blueberry plant, the following has been observed during the sixth fruiting season under the ecological conditions prevailing at the farm of the inventor, which is located in Yanchep Springs, Western Australia.
Plant:
   Plant vigor.—Considered strong. This is in contrast of the closest known variety that being the ‘Sharpeblue’ blueberry plant (unpatented) and whose displayed plant vigor is considered only medium to strong.
   Plant growth habit.—Intermediate to intermediate spreading. This growth habit is distinguishable from the closest known variety noted, above, and whose growth habit is considered merely intermediate.
   One year old shoots.—Color — Green. This color is virtually indistinguishable from the closest known variety noted above.
   One year old shoots.—Internode length — Considered medium. This is in contrast to the closest known variety where this same trait is considered medium to long.
Leaf:
   Leaf length.—Considered average for the species. This is in contrast to the closest known variety where the leaf length is considered medium to long for the species.
   Leaf width.—Medium. This is in contrast to the closest known variety where the leaf width is considered medium to broad.
   Leaf ratio, length versus width.—Considered medium for the species. This is in contrast to the closest known variety where this same trait is considered medium to large.
   Leaf shape.—Considered ovate. This trait is similar to that seen in the closest known variety.
   Leaf color.—Dorsal surface — Green. This color appears similar to the closest known variety.
   Leaf color.—Intensity — Dorsal surface — The green color intensity of the new variety is considered medium to dark. This is in contrast to the light to medium green color as typically expressed by the ‘Sharpeblue’ blueberry plant which is the closest known variety.
   Leaf margin.—Considered entire. This is similar to the trait as expressed by the closest known variety.
Flower:
   Flower buds.—Anthocyanin Coloration — Considered very weak. This growth characteristic is similar to that which is expressed by the closest known variety, noted above.
   Inflorescence.—Length — Considered average for the species. This growth characteristic is similar to that expressed by the closest known variety.
   Flower shape.—Corolla — Urceolate. This growth characteristic is similar to that which is expressed by the closest known variety.
   Flower size.—Corolla Tube — Considered medium to medium large. This growth characteristic is similar to that which is expressed by the ‘Sharpeblue’ blueberry plant [unpatented].
   Flower.—Anthocyanin Coloration of Corolla Tube — Considered very weak to weak. This is in contrast to the growth characteristic as expressed by the closest known variety where the same trait is considered to be merely weak.
   Flower.—Corolla Tube Ridges — Present. This is similar to the growth characteristic as expressed by the closest known variety.
Fruit:
   Fruit cluster density.—The present variety has a medium to somewhat dense fruit cluster. This is in contrast to the closest known variety where the fruit cluster density is considered dense to very dense.
   Color intensity.—Unripe Fruit — Considered a medium green. This is in contrast to the closest known variety where this same plant trait is considered light to medium green.
   Fruit size.—Generally — Considered very large for the species. This is in contrast to the fruit produced by the closest known variety which is considered average for the species.
   Fruit shape.—Longitudinal Sectional View — Considered oblate. This is similar to the growth characteristic as expressed by the closest known variety.
   Sepal attitude.—Generally speaking it is considered to be erect to semi-erect. This is in contrast to the same growth characteristic as expressed by the ‘Sharpeblue’ blueberry plant which is [unpatented] and which is the closest known variety, and which further displays a more erect sepal attitude.
   Sepal form.—The present variety displays an incurving sepal form. This is in contrast to the same growth characteristic as expressed by the closest known variety which is considered to have a straight sepal.
   Calyx basin.—Diameter — Considered average for the species. This is in contrast to the same growth characteristic as expressed by the closest known variety and where the Calyx Basin Diameter is considered small to average for the species.
   Calyx basin depth.—Considered medium. This is similar to the growth characteristic as expressed by the closest known variety.
   Intensity of bloom.—Considered strong to very strong. This is in contrast to the same trait as seen in the closest known variety and when the bloom is considered merely strong.
   Skin color.—Dark blue. This color is similar to that as seen in the fruit produced by the closest known variety.
   Fruit firmness.—Generally — The present variety produces fruit which are considered firm to very firm. This is in contrast to the fruit produced by the closest known variety where the fruit is considered to be soft to medium in firmness.
   Fruit sweetness.—Generally — The present variety produces fruit which have a high degree of sweetness. This is in contrast to the closest known variety where the fruit has a sweetness which is only considered average.
   Fruit acidity.—Generally — Considered low for the species. This is in contrast to the closest known variety where the fruit acidity is considered average.
   Fruiting type of plant.—Generally speaking, one year old and current season shoots produce fruit. This is similar to the growth trait as expressed in the closest known variety.
   Vegetative bud burst.—Timing — Considered very early for the species. This is in contrast to the same characteristic as expressed by the closest known variety and where the vegetative bud burst is only considered early for the species.
   Time of flowering.—One Year Old Shoots — The flowering is considered very early for the species. This is in contrast to the same growth characteristic as seen on the closest known variety and where the flowering time is considered only early.
   Flowering time.—Current Year's Shoots — Considered very early for the species. This is in contrast to the earlier time period for flowering on current season shoots for the closest known variety.
   Fruit ripening.—One Year Old Shoots — Generally speaking, the fruit of the new variety begins to ripen very early for this species. This is in contrast to the early to average ripening date for fruit which are produced by the closest known variety.
   Fruit ripening on current year's shoots.—Considered very early for the species. This is in contrast to the early to medium fruit ripening date for the closest known variety.
   Resistance to insects and disease.—No particular susceptibilities were noted. The present variety has not been tested to expose or detect any susceptibilities or resistances to any known plant and/or fruit diseases.

Although the new variety of blueberry plant possesses the described characteristics when grown under the ecological conditions prevailing near Yanchep Springs, Western Australia, it should be understood that the variations of the usual magnitude and characteristics incident to changes in growing conditions, fertilization, pruning, pest control, frost, climatic variables and horticultural management are to be expected.

Having thus described and illustrated my new variety of blueberry plant, what I claim is new and desire to secure by plant Letters Patent is:

1. A distinct variety of blueberry plant, substantially as illustrated and described, and which is characterized principally as to novelty by producing an attractively colored, extra large sized fruit, which has a very good fruit flavor and earlier flowering and fruit production dates when grown under the ecological conditions prevailing near Yanchep Springs, Western Australia.
